(19) United States
(12) Patent Application Publication
(10) Pub. No.: US 2026/0108438 A1
(43) Pub. Date: Apr. 23, 2026

(54) METHOD AND PROCESS FOR MAKING TATTOO STENCIL INK

(71) Applicant: TROY Group, Inc., Costa Mesa, CA (US)

(72) Inventors: Xiaorong Cai, Mount Pleasant, SC (US); Melissa Marie Wolfe, Bridgeport, OH (US); Madisyn Lloyd, Moundsville, WV (US); Carrie A. Gilson, Wheeling, WV (US); Michael R. Riley, Steubenville, OH (US); Brian Dirk, Newport Beach, CA (US); Francis Goossens, Wheeling, WV (US); Kevin L. Heilman, Wheeling, WV (US)

(21) Appl. No.: 19/359,008

(22) Filed: Oct. 15, 2025

Related U.S. Application Data
(60) Provisional application No. 63/708,678, filed on Oct. 17, 2024.

(51) Int. Cl. A61K 8/31; A61Q 1/02; C09D 11/033; C09D 11/037; C09D 11/328; C09D 11/38

(52) U.S. Cl. CPC A61K 8/31; A61Q 1/025; C09D 11/033; C09D 11/037; C09D 11/328; C09D 11/38; A61K 2800/432; A61K 2800/87

(57) ABSTRACT

An ink composition for a tattoo stencil and methods of making the same are disclosed herein. The ink composition comprises a plurality of cationic violet dyes in combination with a humectant, optionally along with surfactants and/or alcohol. It exhibits enhanced color vibrancy and color reproduction of the transferred tattoo design. The disclosed ink composition also exhibits excellent stability under thermal conditions and is suitable for long-term storage.

### CROSS REFERENCE TO RELATED APPLICATION

This application claims the benefit of priority under 35 U.S.C. § 119(e) to U.S. Application No. 63/708,678, filed Oct. 17, 2024, which is incorporated herein by reference in its entirety.

### BACKGROUND

Ink compositions for tattoo stencils and temporary tattoos have become a popular solution for printing on porous and soft substrates like skin. These ink compositions may be developed for use in inkjet printers. The printers print the tattoo ink on stencil paper. The stencil paper is an intermediate step and is used to ultimately transfer the ink onto the skin. There are several inkjet Tattoo inks and products on the market, however, most of the products are unreliable, and the ink clogs inkjet printheads over time. This makes the ink jet printer unusable for long-term use.

While versatile, ink compositions that possess the desired properties such as color vibrancy and accurate color reproduction for optimal transfer of the stencil design onto the target substrate, along with high print quality, ink robustness, and compatibility with inkjet printers, are difficult to achieve. Therefore, a demand for an ink composition to have these properties poses new challenges for the ink developers. The challenge to achieve a balance of the aforementioned properties while maintaining ink reliability and consistent performance in inkjet printing systems is tough to overcome.

Thus, an ink formulation that provides superior color properties and transfer fidelity, while providing long-term stability, is desired. This document describes methods, compositions, and articles that address some or all of the problems described above.

### SUMMARY

The present document is related to an ink composition for a tattoo comprising: a plurality of cationic violet dyes; a humectant; and a surfactant. In some embodiments, the ink composition further comprises an alcohol and water. In some embodiments, the plurality of cationic violet dyes are present in an amount ranging from about 1 wt % to about 10 wt %, based on total weight of the ink composition. In some embodiments, the alcohol is present in an amount up to 15 wt %, based on total weight of the ink composition.

In some embodiments, the humectant comprises trimethylolpropane, 2-pyrrolidinone, glycerol, or a combination thereof. In some embodiments, the humectant comprises: trimethylolpropane, present in an amount ranging from about 5 wt % to about 15 wt %, based on total weight of the ink composition; 2-pyrrolidinone, present in an amount ranging from about 5 wt % to about 15 wt %, based on the total weight of the ink composition; and glycerol, present in an amount ranging from about 5 wt % to about 15 wt %, based on the total weight of the ink composition. In some embodiments, the humectant comprises trimethylolpropane, 2-pyrrolidinone, and glycerol, present in a weight ratio of about 2:1:2.

In some embodiments, the cationic violet dyes comprise a triphenylmethane dye.

In some embodiments, the cationic violet dyes comprise Basic Violet 1, Basic Violet 2, Basic Violet 3, Basic Violet 4, Basic Violet 10, Basic Violet 11:1, Basic Violet 14, Basic Violet 16, or a combination thereof.

In some embodiments, the cationic violet dyes comprise Basic Violet 1, Basic Violet 3, Basic Violet 4, or a combination thereof. In some embodiments, the plurality of cationic violet dyes comprise: Basic Violet 1, present in an amount ranging from about 2 wt % to about 6 wt %, based on total weight of the ink composition; Basic Violet 3, present in an amount ranging from about 2 wt % to about 6 wt %, based on the total weight of the ink composition; and Basic Violet 4, present in an amount ranging from about 2 wt % to about 6 wt %, based on the total weight of the ink composition. In some embodiments, the cationic violet dyes comprise Basic Violet 1, Basic Violet 3, and Basic Violet 4, present in a weight ratio of about 2:1:1.

In another aspect, the present document is related to a method of making an ink composition for a tattoo stencil, the method comprising: preparing a solution comprising a humectant, a surfactant, an alcohol, and water; mixing a plurality of cationic violet dyes in the solution until the solution is substantially homogeneous; and filtering the solution through a filter to remove substantially any undissolved dye, impurities or particulate matter.

In some embodiments, the mixing the cationic violet dye comprises: mixing a first cationic violet dye into the solution; and after the first cationic violet dye is substantially dissolved, mixing a second cationic violet dye into the solution until the second cationic violet dye is substantially dissolved. In some embodiments, after the second cationic violet dye is substantially dissolved, the method further comprises mixing a third cationic violet dye into the solution until the third cationic violet dye is substantially dissolved. In some embodiments, the method comprises adding the first cationic violet dye, the second cationic violet dye, and the third cationic violet dye in a weight ratio of about 2:1:1.

In some embodiments, the humectant is added in an amount ranging from about 20 wt % to about 40 wt %, based on total weight of the ink composition; the surfactant is added in an amount ranging from about 0.05 wt % to about 1 wt %, based on the total weight of the ink composition; and the alcohol is added in an amount up to 15 wt %, based on the total weight of the ink composition. In some embodiments, the humectant comprises trimethylolpropane, 2-pyrrolidinone, and glycerol in a weight ratio of about 2:1:2.

In another aspect, the present document is related to a tattoo stencil, comprising: a substrate comprising a stencil paper; and a tattoo design printed on the substrate, wherein the tattoo design comprises the ink composition of the present disclosure.

In another aspect, the present document is related to a method of forming a tattoo stencil, the method comprising: by a printhead of an inkjet printer, applying ink to a stencil paper substrate to form a tattoo design on the substrate, wherein the ink comprises the ink composition of the present disclosure.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 shows a flow chart describing a method of producing the ink compositions of the present disclosure.

FIG. 2 shows a side-perspective view of an embodiment of an ink composition of the present disclosure printed on a substrate.

FIGS. 3A and 3B show a comparison of transferred ink composition for a (3A) conventional ink formulation and (3B) an ink composition of the present disclosure.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” (or “comprises”) means “including (or includes), but not limited to.”

In this document, when terms such as “first” and “second” are used to modify a noun, such use is simply intended to distinguish one item from another, and is not intended to require a sequential order unless specifically stated. The term “about” or “approximately,” when used in connection with a numeric value, is intended to include values that are close to, but not exactly, the number. For example, in some embodiments, the term “about” or “approximately” may include values that are within +/−10 percent of the value.

The ink compositions for a tattoo stencil in the current disclosure provide enhanced properties for direct transfer to skin. The inks surpass current commercially available ink compositions for temporary tattoos by achieving better transfer fidelity to the skin while providing superior color vibrancy and reliability by utilizing a plurality of cationic violet dyes in combination with a unique humectant package. When a tattoo design on is reproduced on the transfer substrate, e.g., dermal surface, with minimal cracking and fading of the ink, the ink formulation is said to have high transfer fidelity.

This blend of properties is quite difficult to obtain, but with a unique formulation process utilizing good ratios/quantities of multiple cationic inks and an optimized humectant package, the inventors have found that it is obtainable.

The inks are prepared by combining disclosed components, e.g., a plurality of cationic violet dyes, a humectant, a surfactant, and alcohol mixed in water to form an ink composition with enhanced properties and improved reliability. Most existing state-of-the-art ink compositions for a tattoo stencil or temporary tattoo are formulated with a single cationic violet dye, which results in a lower color vibrancy, transfer fidelity, and reliability. For instance, commercially available ink formulations clog the inkjet printhead over time, rendering the inkjet printer unsuitable for long-term use, requiring constant maintenance and head cleaning.

In some embodiments, an ink composition for a tattoo stencil may comprise two or more, three or more, or any plurality of cationic violet dyes, a humectant, a surfactant, and an alcohol in a water-based system. In some embodiments, the plurality of cationic violet dyes are present in an amount ranging from about 1 wt % to about 10 wt %, about 2 wt % to about 8 wt %, about 4 wt % to about 6 wt %, about 2 wt %, about 3 wt %, about 4 wt %, about 5 wt %, about 6 wt %, about 7 wt %, about 8 wt %, about 9 wt %, about 10 wt %, or between any two of the aforementioned values based on the total weight of the ink composition.

In some embodiments, the cationic violet dyes may comprise a triphenylmethane dye. In some embodiments, the cationic violet dye may belong to the Basic Violet dye class, as defined by their Color Index (C.I.) names. In some embodiments, the cationic violet dyes may comprise Basic Violet 1, Basic Violet 2, Basic Violet 3, Basic Violet 4, Basic Violet 10, Basic Violet 11:1, Basic Violet 14, Basic Violet 16, or a combination thereof. In some embodiments, any combination of 2 or more of the aforementioned cationic violet dyes may be utilized in the disclosed ink composition. In some embodiments, the plurality of cationic violet dyes in an embodiment of the disclosed ink composition may comprise Basic Violet 1, present in an amount ranging from about 2 wt % to about 6 wt %, based on total weight of the ink composition, Basic Violet 3, present in an amount ranging from about 2 wt % to about 6 wt %, based on the total weight of the ink composition, and Basic Violet 4, present in an amount ranging from about 2 wt % to about 6 wt %, based on the total weight of the ink composition.

In some embodiments, when multiple cationic violet dyes are used, the cationic violet dyes may be present in any weight ratio of (1 to 10):(1 to 10):(1 to 10). For instance, when Basic Violet Dye 1, Basic Violet Dye 3, and Basic Violet Dye 4 are used, the cationic violet dyes may be present in a weight ratio ranging from about 1:1:8 to about 8:1:1, including but not limited to about 2:1:1, about 1:2:1, or about 1:1:2.

In some embodiments, an ink composition for a tattoo stencil may comprise a humectant present in an amount ranging from about 20 wt % to about 40 wt %, about 25 wt % to about 30 wt %, about 30 wt % to about 35 wt %, about 35 wt % to about 40 wt %, about 40 wt % to about 45 wt %, about 45 wt % to about 50 wt %, or between any two of the aforementioned values based on the total weight of the ink composition.

In some embodiments, the humectant may include glycerol, glycols, lactams, diols, triols, and alcohols. In some embodiments, the humectant may be a humectant package comprising a one or more compounds comprising trimethylolpropane, 2-pyrrolidinone, glycerol, or a combination thereof. In some embodiments, the humectant may comprise trimethylolpropane, present in an amount ranging from about 5 wt % to about 15 wt %, based on total weight of the ink composition, 2-pyrrolidinone present in an amount ranging from about 5 wt % to about 15 wt %, based on the total weight of the ink composition, glycerol, present in an amount ranging from about 5 wt % to about 15 wt %, based on the total weight of the composition.

In some embodiments, when multiple compounds for the humectant are used, each component in of the humectant may be present in any weight ratio of (1 to 10):(1 to 10):(1 to 10). For instance, when trimethylolpropane, 2-pyrrolidinone, and glycerol are used, each component may be present in a weight ratio ranging from about 1:1:8 to about 8:1:1, including but not limited to about 2:1:1, about 1:2:1, about 2:1:2, or about 1:1:2.

In some embodiments, an ink composition for a tattoo stencil may comprise a surfactant present in an amount ranging from about 0.05 wt % to about 1 wt %, about 0.1 wt % to about 1 wt %, about 0.2 wt % to about 0.8 wt %, about 0.4 wt % to about 0.6 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, or between any two of the aforementioned values based on the total weight of the ink composition. In some embodiments, the surfactant may be non-ionic. In some embodiments, the surfactant may comprise commercially available surfactants, including but not limited to, fluorosurfactants, such as Capstone surfactants from DuPont, APFS surfactants from Advanced Polymer, Inc., Chemguard from Chemguard Specialty Chemical, Nevec from 3M, alkylaryl polyether alcohol nonionic surfactants, such as octylphenoxy-polyethoxyethanol available from Dow Chemical Company of Midland, MI under the TRITON X series of trade names; alkylamine ethoxylates nonionic surfactants such as from Dow Chemical Company under the TRITON FW series, TRITON CF-10, TERGITOL trade names; ethoxylated acetylenic diol surfactants available from Air Products and Chemicals, Inc. of Allentown, PA under the SURFYNOL and Dynol trade names; polysorbate products available from ICI Chemicals & Polymers Ltd. of Middlesborough, UK under the trade name TWEEN; polyalkylene and polyalkylene modified surfactants available from Crompton OSI Specialties of Greenwich, CT, under the trade name SILWET, polydimethylsiloxane copolymers and surfactants available from Crompton OSI Specialties under the trade name COATOSIL; alcohol alkoxylates nonionic surfactants available from Uniqema of New Castle, DE, under the trade names RENEX, BRIJ, and UKANIL; sorbitan ester products available from Omya Peralta GmbH of Hamburg, Germany under the trade names SPAN and ARLACEL; alkoxylated esters/polyethylene glycol surfactants available from ICI Chemicals & Polymers Ltd. under the trade names TWEEN, ATLAS, MYRJ and CIRRASOL; alkyl phosphoric acid ester surfactant products such as amyl acid phosphate available from Chemron Corporation of Paso Robles, CA, under the trade name CHEMPHOS TR-421; alkyl amine oxides available from Chemmron Corporation under the CHEMOXIDE series of surfactants; anionic sarcosinate surfactants available from Hampshire Chemical Corporation of Nashua, NH under the HAMPOSYL series of surfactants; glycerol esters or polyglycol ester nonionic surfactants available from Calgene Chemical Inc. of Skokie, IL under the HODAG series of surfactants, available from Henkel-Nopco A/S of Drammen, Norway under the trade name ALPHENATE, available from Hoechst AG of Frankfurt, Germany under the trade name SOLEGAL W, and available from Auschem SpA of Milan, Italy under the trade name EMULTEX; polyethylene glycol ether surfactants available from Takemoto Oil and Fact Co. Ltd. of Japan under the trade name NEWKALGEN; modified poly-dimethyl-silicone surfactants available from BYK Chemie of Wesel, Germany under the BYK 300 series of surfactants; and other commercially available surfactants known to those skilled in the art. Particularly desirable surfactants are non-ionic surfactants.

In some embodiments, an ink composition for a tattoo stencil may comprises alcohol present in an amount ranging from about 0 wt % to about 15 wt %, about 2 wt % to about 15%, about 4 wt % to about 14%, about 6 wt % to about 12 wt %, about 8 wt % to about 10%, about 0 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, or between any two of the aforementioned values based on the total weight of the ink composition. In some embodiments, the alcohol may be isopropyl alcohol, ethyl alcohol, or a combination thereof.

In some embodiments, an ink composition for a tattoo stencil may comprise a biocide present in an amount ranging up to about 0.15 wt %. In some embodiments, the biocide may comprise commercially available biocides, including but not limited to Phenoxyethanol, Proxel™ GXL, KATHON™, ROCIMA™, UCARCIDE™, BIOBAN™, or a combination thereof.

Another aspect of the present disclosure relates to a method for making an ink composition for a tattoo stencil or an ink composition for a temporary tattoo. In some embodiments, the ink composition may be prepared by making a solution comprising one or more of a humectant, a surfactant, alcohol, and water in an amount and/or ratio disclosed above. Next, a plurality of cationic violet dyes in an amount and/or ratio disclosed above are mixed into the solution until the solution is substantially homogeneous. As used herein, the term “substantially” refers to the complete or nearly complete extent or degree of an action, characteristic, property, state, structure, item, or result. For example, a solution that is “substantially” homogeneous means that the solution is either completely or nearly completely free of undissolved dye, impurities, or other particulate matter. In some embodiments, undissolved dye, impurities, or other particulate matter may be present in an amount less than 5 wt %, less than 4 wt %, less than 3 wt %, less than 2 wt %, less than 1 wt %, or less than 0.5 wt %, based on the total weight of the solution.

By mixing the cationic violet dye components into the composition after other ingredients are mixed first, the formation of localized high-concentration of particulates is avoided, which could lead to adverse reaction and prevent “ink crashes.” This can avoid or reduce clogging of inkjet nozzles when the ink composition is incorporated in an inkjet print device that is used for printing tattoo stencils and temporary tattoos. In some embodiments, the mixing of the components may be carried out using any common laboratory mixing instrument including but not limited to a liquid mixer agitator or any types of overhead mixers. In some embodiments, each component may be mixed at a temperature ranging from about 19° C. to about 27° C., for a period of at least 30 minutes or until the cationic violet dye is substantially dissolved.

In some embodiments, when multiple cationic violet dyes are mixed, each cationic violet dye component is added to the mixture sequentially, only after each component is substantially dissolved to form a substantially homogeneous solution. For instance, after a solution of the humectant, surfactant, alcohol, and water is prepared, the first cationic violet dye is incorporated into the solution until the first cationic violet dye is substantially dissolved. Afterwards, a second cationic violet dye is incorporated in the solution until the second cationic violet dye is substantially dissolved. Afterwards, a third cationic violet dye is incorporated in the solution until the third cationic violet dye is substantially dissolved.

After all components are dissolved and the resulting ink composition solution is substantially homogeneous, the ink composition solution is filtered through a size exclusion filter such as a membrane filter, to remove substantially undissolved dye, impurities, or particulate matter from the solution. In some embodiments, the filter may have a pore size of 1.0 micron rating, meaning that only particles smaller than 1.0 micron will pass through the filter.

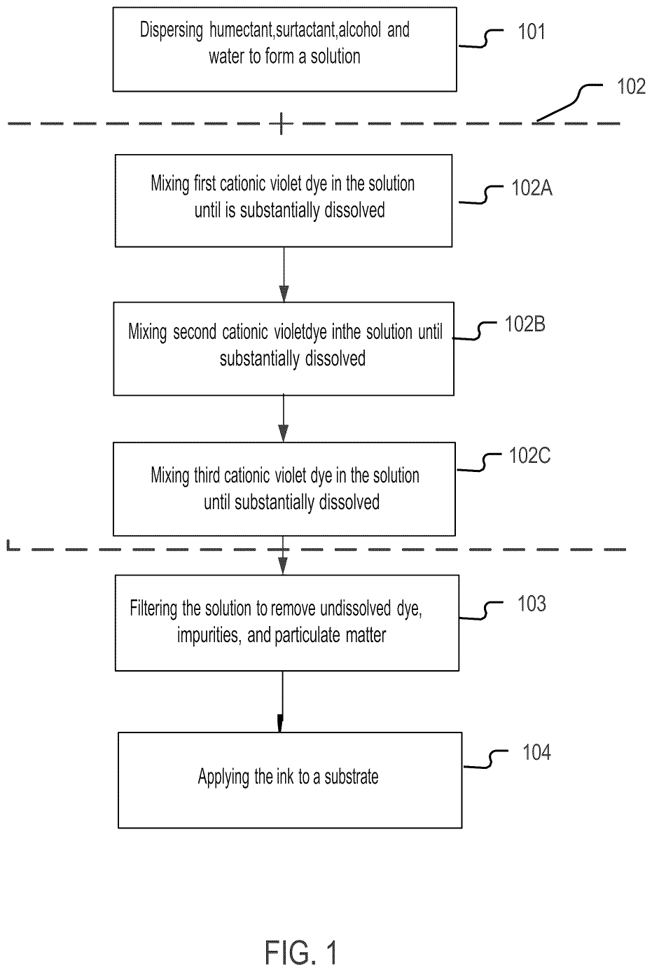

Referring to FIG. 1, a flow chart for making and using an ink composition for a tattoo stencil of the present disclosure is shown. As seen in FIG. 1, step 101 may include dispersing one or more of humectant, a surfactant, an alcohol, and water in an amount and/or ratio disclosed above to form a solution. Step 102 is the addition of a plurality of cationic violet dyes to the solution until the dyes are substantially dissolved. Optionally, step 102 may include sub-steps 102A, 102B, and/or 102C, comprising mixing each individual cationic violet dye in an amount and/or ratio disclosed above in sequence, until the solution is substantially homogeneous. In 102A, a first cationic violet dye is mixed in the solution until the first cationic violet dye is substantially dissolved in the solution, forming a homogeneous solution. Afterwards, in 102B, a second cationic violet dye is incorporated in the solution until the second cationic violet dye is substantially dissolved. Optionally, in 102C, a third cationic violet dye is incorporated in the solution until the third cationic violet dye is substantially dissolved. Similar additional substeps may follow if additional cationic violet dyes are included in the composition.

After the solution is prepared in 103, the homogeneous ink composition solution is filtered through a filter to remove substantially undissolved dye, impurities, or particulate matter. In 104, the filtered homogenous ink composition is loaded into a printhead device such as an inkjet printer and printed on a print substrate.

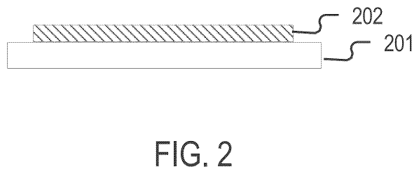

Another aspect of the present disclosure relates to a tattoo stencil comprising a substrate comprising stencil paper, and a tattoo design printed on the substrate using an ink composition of the present disclosure. In another aspect, the present disclosure relates to a method of forming a tattoo stencil. In some embodiments, the tattoo stencil may be formed by applying an ink composition of the present disclosure to a stencil paper substrate using a printhead of an inkjet printer. Referring to FIG. 2, an ink composition of the present disclosure (e.g., 202) printed on a substrate 201 is shown. In some embodiments, the substrate may be a commercial stencil paper or inkjet stencil tracing paper. In some embodiments, the stencil paper is designed to effectively transfer printed designs onto another surface, e.g., the skin.

The resulting ink composition exhibits better transfer fidelity to the skin while providing superior color vibrancy and enhanced properties for direct transfer on skin. The inks surpass current commercially available ink compositions for temporary tattoos by providing superior color vibrancy and robustness (i.e., more complete ink transfer from the stencil to the transfer substrate). Moreover, the disclosed ink composition is more reliable for use in conventional inkjet printers and current inkjet printing technology by mitigating clogging of the inkjet printhead.

Additional advantages of the disclosed technology will become readily apparent to those skilled in the art from the following detailed description, wherein only some embodiments are shown and described. As will be realized, the disclosed technology is capable of other and different embodiments, and its details are capable of modifications in various obvious respects, all without departing from the disclosed technology. Accordingly, the examples and descriptions are to be regarded as illustrative in nature and not as restrictive.

### EXAMPLES

The methods presented in this document are next described by means of the following example. The use of these and other examples anywhere in the specification is illustrative only, and in no way limits the scope and meaning of the invention or of any exemplified form. Likewise, the invention is not limited to any particular embodiment described herein. Indeed, modifications and variations of the invention may be apparent to those skilled in the art upon reading this specification and can be made without departing from its spirit and scope. The invention is therefore to be limited only by the terms of the claims, along with the full scope of equivalents to which the claims are entitled.

### Example 1: Preparation of Ink Composition for a Tattoo Stencil/Temporary Tattoo

TABLE 1
Multi-Cationic Violet Ink Composition Formulations

| | Weight % in the Formulation | | | | | |
|---|---|---|---|---|---|---|
| Ingredient | Ink 1 | Ink 2 | Ink 3 | Ink 4 | Ink 5 | Ink 6 |
| Basic Violet 1 | 4 | 0 | 0 | 2 | 2 | 2 |
| Basic Violet 3 | 0 | 4 | 0 | 1 | 2 | 0 |
| Basic Violet 4 | 0 | 0 | 4 | 1 | 0 | 2 |
| Humectant 1 | 10 | 10 | 10 | 10 | 10 | 10 |
| (Trimethylolpropane) | | | | | | |
| Humectant 2 | 5 | 5 | 5 | 5 | 5 | 5 |
| (2-Pyrrolidinone) | | | | | | |
| Humectant 3 | 10 | 10 | 10 | 10 | 10 | 10 |
| (Glycerol) | | | | | | |
| Alcohol | 5 | 5 | 5 | 5 | 5 | 5 |
| (Isopropanol) | | | | | | |
| Surfactant | 0.5 | 0.5 | 0.5 | 0.5 | 0.5 | 0.5 |
| (Surfynol 465) | | | | | | |
| Biocide | 0.15 | 0.15 | 0.15 | 0.15 | 0.15 | 0.15 |
| D.I. Water | Balanced | Balanced | Balanced | Balanced | Balanced | Balanced |

As seen in Table 1, six ink formulations (i.e., Ink 1, Ink 2, Ink 3, Ink 4, Ink 5, and Ink 6) of the present disclosure were prepared using the method disclosed above. Ink 1, Ink 2, and Ink 3 each comprised one cationic violet dye (Basic Violet 1, Basic Violet 3, and Basic Violet 4, respectively) at 4 wt %. Ink 4 comprised a combination of three cationic violet dyes at a 2:1:1 weight ratio of Basic Violet 1, Basic Violet 3, and Basic Violet 4, each at 2 wt %, 1 wt %, and 1 wt %, respectively. Ink 5 comprised a 1:1 ratio of two cationic violet dyes, Basic Violet 1 and Basic Violet 3, each at 2 wt %, while Ink 6 comprised a 1:1 ratio of two cationic violet dyes, Basic Violet 1 and Basic Violet 4, each at 2 wt %. All ink formulations comprised a humectant package comprising 10 wt % trimethylolpropane, 5 wt % 2-Pyrrolidinone, and 10 wt % glycerol, isopropanol at 5 wt %, Surfynol 465 surfactant at 0.5 wt %, and a biocide at 0.15 wt %, all based on the total weight of the ink composition.

### Example 2: Evaluation of the Stability of the Ink Compositions

To assess ink stability, an accelerated aging test was conducted by storing the ink samples of Example 1 in an oven at 60° C. for 12 weeks. After this period, the inks were loaded into the printers and printed on the tattoo stencil substrate. Print quality of the ink compositions was evaluated using both thermal and Piezo inkjet printing technology using the HP® OfficeJet 200 Mobile Printer and the Epson® Workforce Supertank ST-M1000 Printer, respectively. Both print platforms demonstrated excellent print quality on a tattoo stencil substrate with respect to color vibrancy and color reproduction. These results indicate strong thermal stability of the ink compositions and are suitable for long-term storage (up to three years) under storage conditions between about 50° F. and about 90° F. and a humidity level between about 40% and about 60%.

### Example 3: Evaluation of the Color Vibrancy and Transfer Fidelity of the Ink Compositions

Two temporary tattoo samples were prepared by printing a tattoo design on a stencil paper substrate with Ink 1 and Ink 4 of Table 1. The target transfer skin area was prepared by shaving, cleaning, and disinfecting the target area. A stencil solution was applied to the skin to facilitate the transfer of the design. In some embodiments, a stencil solution may comprise glycol such as propylene glycol. Afterwards, the printed design was positioned and pressed firmly onto the prepared area for a few seconds. The stencil paper substrate was then removed to reveal the transferred image.

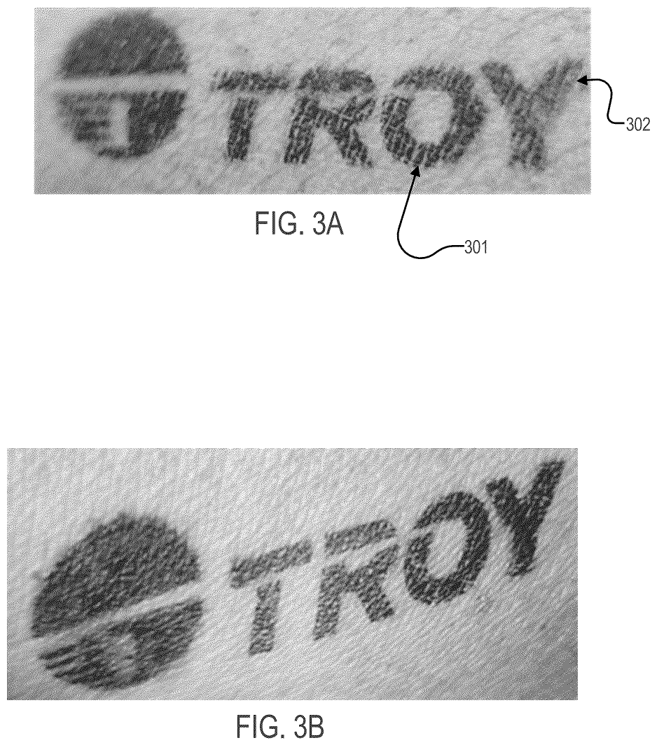

Referring now to FIGS. 3A and 3B, color transfer results of Ink 1 (FIG. 3A) and Ink 4 (FIG. 3B) are shown. As seen in FIG. 3A, Ink 1 exhibited severe ink cracking 301 on the skin and fading 302 around the design edge, indicating incomplete ink transfer and suboptimal transfer fidelity. Moreover, the color was not vibrant, and color reproduction was inconsistent. In comparison, the reproduction of Ink 4 in FIG. 3B exhibited no ink cracking or fading upon skin transfer, indicating more complete ink transfer and superior transfer fidelity, and the ink remained vibrant and glossy.

These results show that compared to an ink composition having a single cationic violet dye, an ink composition having multiple cationic violet dyes exhibited superior color vibrancy and reproduction, and better transfer on the skin.

Overall, the disclosed ink composition formulation comprising multiple cationic violet dyes achieves superior color reproduction, vibrancy, and better transfer on the skin, compared to commercially available ink compositions containing a single cationic violet dye. The specific combination of the quantity and the weight ratios of the cationic violet dyes in the disclosed humectant is responsible for the unexpectedly beneficial results presented herein.

As used in this document, the term “printer” refers to an electromechanical device that is configured to apply a marking material to a substrate to print characters and/or images on the substrate. In the context of an inkjet printer, the marking material will be ink. The printer will hold the ink in one or more cartridges, reservoirs, or other containers. The printer will include one or more printheads that, in operation, each will draw ink one or more of the cartridges and transfer the ink to the substrate, such as by spraying droplets of the ink toward the paper.

The features and functions described above, as well as alternatives, may be combined into many other different systems or applications. Various alternatives, modifications, variations, or improvements may be made by those skilled in the art, each of which is also intended to be encompassed by the disclosed embodiments.

1. An ink composition for a tattoo stencil comprising:
a plurality of cationic violet dyes;
a humectant; and
a surfactant.

2. The ink composition of claim 1, further comprising an alcohol and water.

3. The ink composition of claim 1, wherein the plurality of cationic violet dyes are present in an amount ranging from about 1 wt % to about 10 wt %, based on total weight of the ink composition.

4. The ink composition of claim 2, wherein the alcohol is present in an amount up to 15 wt %, based on total weight of the ink composition.

5. The ink composition of claim 1, wherein the humectant comprises trimethylolpropane, 2-pyrrolidinone, glycerol, or a combination thereof.

6. The ink composition of claim 1, wherein the humectant comprises:
trimethylolpropane, present in an amount ranging from about 5 wt % to about 15 wt %, based on total weight of the ink composition;
2-pyrrolidinone, present in an amount ranging from about 5 wt % to about 15 wt %, based on the total weight of the ink composition; and
glycerol, present in an amount ranging from about 5 wt % to about 15 wt %, based on the total weight of the ink composition.

7. The ink composition of claim 6, wherein the humectant comprises trimethylolpropane, 2-pyrrolidinone, and glycerol, present in a weight ratio of about 2:1:2.

8. The ink composition of claim 1, wherein the cationic violet dyes comprise a triphenylmethane dye.

9. The ink composition of claim 1, wherein the cationic violet dyes comprise Basic Violet 1, Basic Violet 2, Basic Violet 3, Basic Violet 4, Basic Violet 10, Basic Violet 11:1, Basic Violet 14, Basic Violet 16, or a combination thereof.

10. The ink composition of claim 1, wherein the cationic violet dyes comprise Basic Violet 1, Basic Violet 3, Basic Violet 4, or a combination thereof.

11. The ink composition of claim 1, wherein the plurality of cationic violet dyes comprise:
Basic Violet 1, present in an amount ranging from about 2 wt % to about 6 wt %, based on total weight of the ink composition;
Basic Violet 3, present in an amount ranging from about 2 wt % to about 6 wt %, based on the total weight of the ink composition; and
Basic Violet 4, present in an amount ranging from about 2 wt % to about 6 wt %, based on the total weight of the ink composition.

12. The ink composition of claim 1, wherein the cationic violet dyes comprise Basic Violet 1, Basic Violet 3, and Basic Violet 4, present in a weight ratio of about 2:1:1.

13. A method of making an ink composition for a tattoo stencil, the method comprising:
preparing a solution comprising a humectant, a surfactant, an alcohol, and water;
mixing a plurality of cationic violet dyes in the solution until the solution is substantially homogeneous; and
filtering the solution through a filter to remove substantially any undissolved dye, impurities or particulate matter.

14. The method of claim 13, wherein mixing the cationic violet dye comprises:
mixing a first cationic violet dye into the solution; and
after the first cationic violet dye is substantially dissolved, mixing a second cationic violet dye into the solution until the second cationic violet dye is substantially dissolved.

15. The method of claim 14, further comprising, after the second cationic violet dye is substantially dissolved, mixing a third cationic violet dye into the solution until the third cationic violet dye is substantially dissolved.

16. The method of claim 13, wherein
the humectant is added in an amount ranging from about 20 wt % to about 40 wt %, based on total weight of the ink composition;
the surfactant is added in an amount ranging from about 0.05 wt % to about 1 wt %, based on the total weight of the ink composition; and
the alcohol is added in an amount up to 15 wt %, based on the total weight of the ink composition.

17. The method of claim 13, wherein the humectant comprises trimethylolpropane, 2-pyrrolidinone, and glycerol in a weight ratio of about 2:1:2.

18. The method of claim 15, wherein the method comprises adding the first cationic violet dye, the second cationic violet dye, and the third cationic violet dye in a weight ratio of about 2:1:1.

19. A tattoo stencil, comprising:
a substrate comprising a stencil paper; and
a tattoo design printed on the substrate, wherein the tattoo design comprises the ink composition of claim 1.

20. A method of forming a tattoo stencil, the method comprising:
by a printhead of an inkjet printer, applying ink to a stencil paper substrate to form a tattoo design on the substrate,
wherein the ink comprises the ink composition of claim 1.
